(19) United States
(12) Patent Application Publication
(10) Pub. No.: US 2005/0273366 A1
(43) Pub. Date: Dec. 8, 2005

(54) Radiographic image detection device and radiographic imaging system

(75) Inventor: Hiromu Ohara, Tokyo (JP)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: KONICA MINOLTA MEDICAL & GRAPHIC, INC.

(21) Appl. No.: 11/125,220

(22) Filed: May 10, 2005

(30) Foreign Application Priority Data
May 13, 2004 (JP) 2004-143574

(51) Int. Cl. G06F 17/60; G21K 1/12; A61B 6/00

(52) U.S. Cl. 705/3

(57) ABSTRACT

A radiographic image detection device of a portable type to detect radiation and obtain image information on a radiographic image, includes: an accompanying information obtaining unit to obtain accompanying information concerning radiographing for obtaining the radiographic image; and a correlating unit to correlate the accompanying information obtained by the accompanying information obtaining unit with the image information obtained through the radiographing.

### BACKGROUND OF THE INVENTION

1. Field of the Invention

The present invention relates to a radiographic image detection device to detect a radiographic image as typified by an X-ray image, and to a radiographic imaging system to take a radiographic image by using the radiographic image detection device.

2. Description of the Related Art

For medical diagnosis, there have been widely used radiographic images obtained by radiating radiation such as X-rays to a subject and detecting intensity distribution of radiation which has passed through the subject. In recent years, there has been proposed a radiographic imaging system using an FPD (Flat Panel Detector) which detects radiation and converts it to an electric signal upon radiographing, and outputs the electric signal as radiographic image information.

As such a radiographic imaging system, there has been known a system configured such that an FPD placed in a radiographic room is connected to a predetermined console such as a PC (Personal Computer) for operating the FPD through a predetermined communication line, so that the degree of freedom of system configuration is increased (for example, see JP-Tokukai-2003-199736A).

In addition, cassette-type FPDs have also been developed for the purpose of improving transportability and handling properties of the FPDs (for example, see JP-Tokukaihei-6-342099A). Moreover, there has also been proposed a system configured to enable a cassette type FPD and a console to communicate various information such as image information on a radiographic image by a wireless system (for example, see JP-Tokukai-2003-210444A). For such an FPD, it has been prosposed to configure the FPD to include an image memory for storing image information obtained by detection, and transmit the image information stored in the image memory to a console in accordance with a transmission instruction sent from the console.

Meanwhile, especially in the medical field, when a radiographic image is taken by radiating radiation to a subject and detecting radiation which has passed through the subject, and when the radiographic image is used for diagnosis, the radiographing date and time is important in some cases.

In view of this, a radiographic imaging system has been proposed, in which image information on an obtained radiographic image is transferred to a console, and, on the console side, accompanying information such as the radiographing date and time is correlated with the transferred image information (for example, see JP-Tokukai-2002-133398A).

However, for example, when a radiographic image detection device of a portable type such as a cassette-type FPD is used for radiographing, considering the communication band, it is not realistic to transfer the image data and the accompanying information to the console, which is an external device, every time radiographing is performed. It is therefore necessary to store the image data obtained by radiographing in the image memory in the main body of the FPD, and then transfer the image data from the FPD to an external device such as the console, or read the image data in the FPD from the console.

In this case, when an image stored in the FPD is to be used after some time has passed since the radiographing, it is impossible in some cases to determine what is imaged in the image data. In particular, regarding a radiographic image detection device of a portable type which can be used in various sites, when image data is stored in the radiographic image detection device, it is even more difficult to determine what is imaged in the image data.

Furthermore, although it is necessary to distinguish a plurality of image data from each other after performing radiographing for a plurality of times, it has been difficult to distinguish them based on the images only.

### SUMMARY OF THE INVENTION

The present invention is made to solve the above-described problems. An object of the invention is to provide a radiographic image detection device and a radiographic imaging system which are capable of always grasping accompanying information to accompany a radiographic image accurately, with respect to a portable-type FPD used in various sites.

In order to attain the above object, a radiographic image detection device according to a first aspect of the invention is a radiographic image detection device of a portable type to detect radiation and obtain image information on a radiographic image, comprising: an accompanying information obtaining unit to obtain accompanying information concerning radiographing for obtaining the radiographic image; and a correlating unit to correlate the accompanying information obtained by the accompanying information obtaining unit with the image information obtained through the radiographing.

Thus, since it is possible to correlate the accompanying information with the image information, even when image data is stored in the radiographic image detection device of a portable type which can be used in various sites, what is imaged in the image data is identified immediately.

Moreover, it is preferred that the correlating unit comprises an image memory capable of storing a plurality of image information, and correlates each accompanying information with corresponding one of the plurality of image information stored in the image memory.

Thus, even when radiographing is performed for a plurality of times, since each accompanying information is correlated with the image information corresponding thereto, it is easy to identify the plurality of image data.

It is preferred that the radiographic image detection device further comprises a free space detecting unit to detect a free space in the image memory; and a radiographing possibility judging unit to judge a possibility of next radiographing based on whether or not a total information amount of the image information, the accompanying information and correlation information for correlating the accompanying information with the image information exceeds the free space in the image memory detected by the free space detecting unit.

Since the radiographing possibility judging unit judges the possibility of next radiographing in consideration of the information amount of not only the image information but those of the accompanying information and the correlation information, it is possible to accurately judge whether the next radiographing is possible in a case where the accompanying information is correlated with the image information to be stored.

Moreover, it is preferred that there is further comprised a radiographing instruction receiving unit to receive a radiographing instruction from an external device, wherein the correlating unit correlates the accompanying information with the image information based on the radiographing instruction.

Thus, since the accompanying information is correlated with the image information based on the radiographing instruction, the operational properties of the radiographic image detection device are improved.

It is preferred that the accompanying information is radiographing date and time, and the accompanying information obtaining unit obtains the accompanying information based on the radiographing instruction, prior to obtaining the image information on the radiographic image.

It is necessary to obtain as accurate radiographing date and time as possible. Meanwhile, a little period of time is required for obtaining the image information on the radiographic image. Therefore, if the date and time information which is accompanying information is obtained after obtaining the image information on the radiographic image, date and time deviated a little from the radiographing timing would be obtained. It is possible to prevent such a situation and obtain accurate radiographing date and time by obtaining the accompanying information prior to obtaining the image information on the radiographic image.

It is preferred that there is further comprised a communication unit to receive the radiographic instruction from the external device through wireless communication, wherein the communication unit receives the accompanying information prior to the radiographing.

Since the radiographic image detection device is a portable type, the radiographic image detection device may be displaced immediately after the radiographing for the subsequent radiographing, and, as a result, the wireless communication environment may be changed. Therefore, if the accompanying information is transmitted after the radiographing, it is difficult to displace the radiographic image detection device immediately after the radiographing for the subsequent radiographing, and thus the operational properties of the radiographic image detection device are deteriorated. It is possible to prevent such a situation by receiving the accompanying information prior to the radiographing.

According to a second aspect of the invention, a radiographic imaging system comprising the radiographic image detection device of the first aspect and a console to operate the radiographic image detection device, wherein each of the radiographic image detection device and the console comprises a communication unit to communicate with an external device, and the console receives, through the communication unit thereof, the image information and the accompanying information correlated with the image information, from the radiographic image detection device.

Thus, since the console receives the image information and the accompanying information correlated with the image information from the radiographic image detection device, what is imaged in the received image information is immediately identified.

According to a third aspect of the invention, a radiographic imaging system comprising the radiographic image detection device of the first aspect and a console to operate the radiographic image detection device, wherein each of the radiographic image detection device and the console comprises a communication unit to communicate with an external device, and the console instructs, through the communication unit thereof, the radiographic image detection device to transmit the image information and the accompanying information correlated with the image information, to the console.

Thus, since the console instructs the radiographic image detection device to transmit the image information and the accompanying information correlated with the image information, what is imaged in the image information transmitted from the radiographic image detection device is immediately identified.

According to a fourth aspect of the invention, a radiographic imaging system comprising the radiographic image detection device of the first aspect and a radiographing operation device to operate the radiographic image detection device, wherein each of the radiographic image detection device and the radiographing operation device comprises a communication unit to communicate with an external device, the radiographing operation device transmits, through the communication unit thereof, a radiographing instruction signal to the radiographic image detection device, and the radiographic image detection device correlates the accompanying information with the image information based on the radiographing instruction signal.

Thus, when radiographing operation is performed with the radiographing operation device, the accompanying information is automatically correlated with the image information based on the radiographing instruction sent from the radiographing operation device. Therefore, operational properties of the system are improved.

Moreover, it is preferred that each communication unit performs communication by using a wireless signal.

Since the portable-type radiographic image detection device can communicate with the external device through wireless communication, it is not necessary to connect a cable or the like to the radiographic image detection device. Accordingly, restriction in the radiographing is decreased, and operational properties thereof are improved.

### BRIEF DESCRIPTION OF THE DRAWINGS

Although the present invention will become more fully understood from the detailed description given hereinbelow and the accompanying drawings which are given by way of illustration only, and thus are not intended as a definition of the limits of the present invention, and wherein;

FIG. 1 is a view showing a schematic configuration of a radiographic imaging system shown as an embodiment applying the present invention;

FIG. 2 is a block diagram showing a configuration of a main part of a radiographic image detection device which constitutes the radiographic imaging system of FIG. 1;

FIGS. 3A and 3B are structural views of image files prepared by the radiographic image detection device of FIG. 2;

FIG. 4 is a block diagram showing a configuration of a main part of a console which constitutes the radiographic imaging system of FIG. 1; and

FIGS. 5A to 5C are flowcharts showing an operation of each of the console, a radiographing operation device, and the radiographic image detection device in the radiographic imaging system of FIG. 1 where the devices are in interlinking control, in which FIG. 5A shows an operation of the console, FIG. 5B shows an operation of the radiographing operation device, and FIG. 5C shows an operation of the radiographic image detection device.

### DESCRIPTION OF THE PREFERRED EMBODIMENT

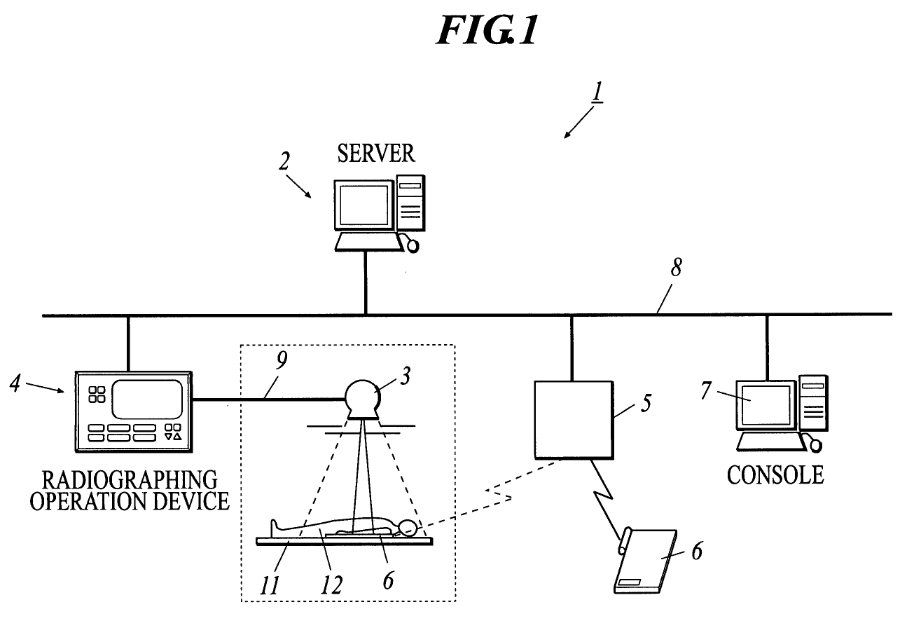

Hereinbelow, an embodiment of the invention will be described with reference to FIGS. 1 to 5C.

FIG. 1 is a view showing a schematic configuration of an embodiment of a radiographic imaging system 1 applying a flat panel detector (hereinafter referred to as “FPD”) 6 of a cassette type as a radiographic image detection device according to the invention.

As shown in FIG. 1, in the radiographic imaging system 1 according to the embodiment, connected through a wired network 8 are: a server 2 to manage information concerning radiographic imaging; a radiographing operation device 4 to operate a radiographic imaging device 3; a base station 5 to perform communication through a wireless LAN by a system such as IEEE802.11b; and a console 7 to transmit information in connection with radiographing to the FPD 6 based on radiographic examination order information stored in the server 2 (to be described later), confirm and input a patient name, a radiographing region and the like at the time of radiographing using the FPD 6, and also check the radiographic image.

The network 8 may be a communication line exclusive to the system; however, the network 8 is more preferably an existing line such as Ethernet (registered trademark), since otherwise the degree of freedom of system configuration would decrease, or for other reasons.

A radiation generating device 3 is connected to the radiographing operation device 4 through a cable 9. The radiation generating device 3 radiates radiation to a subject upon taking a radiographic image thereof.

The server 2 includes a computer. Radiographic examination order information (hereinafter abbreviated to “order information”) based on an examination order by a doctor is stored in the hard disk.

A control unit thereof makes an external storage device or the like store such as the order information inputted from an input operation unit or the like.

Here, the order information is identified based on an ID code (patient ID) for each patient. The order information contains patient information and region information corresponding to each patient ID.

The patient information is information on a patient for specifying a patient 12, such as the name, age, sex, and date of birth of the patient 12.

The region information is information for specifying a radiographing region (part to be radiographed of the body of a person to be radiographed).

The information which is inputted from the input operation unit or the like and stored in an external storage device or the like is not limited to that illustrated above.

The control unit performs correction processing, A/D conversion, normalization processing, gradation processing and the like on image information transmitted from the later-described console 7.

The radiation generating device 3 radiates radiation to the patient 12 who is a subject lying in a bed 11. In a lower portion of the bed 11, there is provided a detection device loading opening (not shown) through which loaded is the FPD 6 to read strength of the radiation and detect a radiographic image.

The radiographing operation device 4 is configured, for example, to permit a radiological technologist to select an examination program and a radiographing program by performing input from an input operation unit (not shown). Moreover, the radiographing operation device 4 receives the order information from the server 2 through the network 8.

The radiographing operation device 4 refers to the order information sent from the server 2, and radiographing conditions such as an X-ray tube potential, a tube current, a focal spot size, an irradiation field, and an exposure time period of the radiation generating device 3 are set based on the region information contained in the order information as well as selected examination program and radiographing program. Thereafter, when the radiological technologist operates the radiographing operation device 4 and makes a radiographing instruction, a radiographing instruction signal is transmitted to the FPD 6 through the wireless LAN. The radiation generating device 3 then radiates radiation to expose the FPD 6.

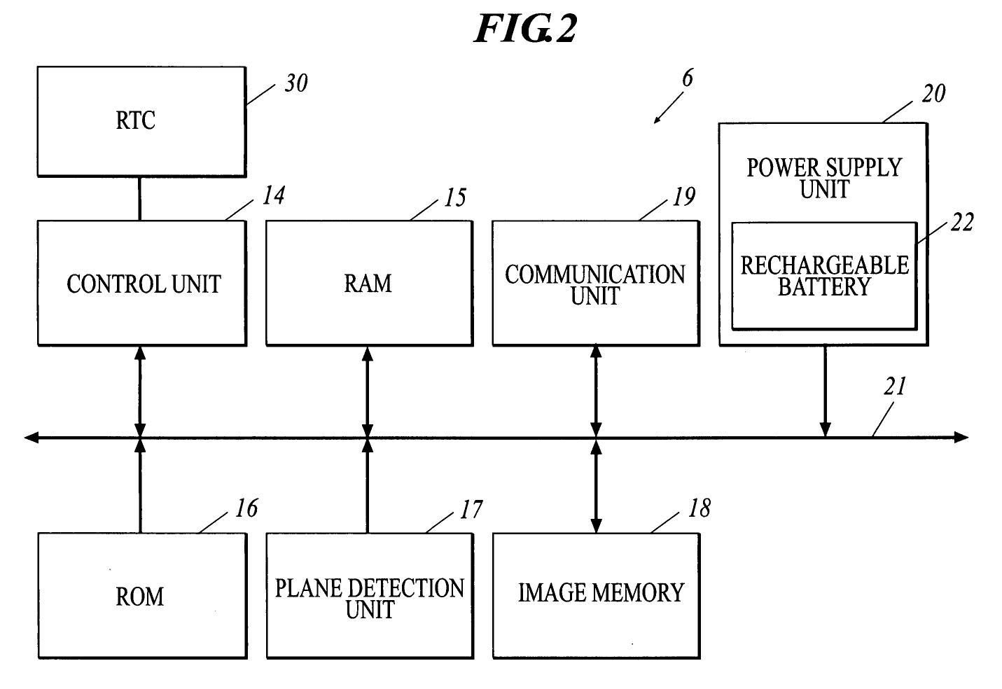

As shown in FIG. 2, the FPD 6 comprises: a control unit 14; a RAM 15; a ROM 16; a plane detection unit 17 to detect radiation; an image memory 18 to store image information; a communication unit 19; a power supply unit 20; and the like. Each unit is connected to each other through a bus 21.

The FPD 6 has a detection device ID number as a detection device identification information unique to the FPD 6, in order to identify the FPD 6 from other FPDs 6. The detection device ID number is stored in the ROM 16 in advance. Note that the detection device ID number may be stored in a storage unit other than the ROM 16.

The plane detection unit 17 includes a first layer to emit light in accordance with strength of the radiation which is made incident thereon, the first layer containing phosphor as a main ingredient. At the back face of the first layer viewing from the radiation incidence side, provided is a second layer to convert the light outputted from the first layer into electric energy. At the back face of the second layer, there is provided a third layer in which arranged in a matrix shape are: capacitors to accumulate the electric energy, which is obtained by converting light by the second layer, in a glass substrate for each pixel; and switching elements such as a TFT (Thin Film Transistor) to output the electric energy accumulated in the capacitors as a signal. Instead of the first and second layers, a radiation/electric energy converting layer to directly convert radiation into electric energy may be provided.

The image memory 18 comprises a non-volatile memory such as a flash memory. The electric signal accumulated in the plane detection unit 17 is read based on a read signal sent from the control unit 14, and stored in the image memory 18 as image information on a radiographic image.

The communication unit 19 is an interface to perform communication of various information between the radiographing operation device 4 and the console 7 through the base station 5, by using the wireless LAN by a system such as IEEE802.11b.

The power supply unit 20 includes a rechargeable battery 22 to supply power to each unit constituting the FPD 6. The power supply unit 20 is configured to be rechargeable through a charging terminal (illustration omitted) provided in a predetermined position of the FPD 6.

The control unit 14 comprises a CPU (Central Processing Unit) and the like. The control unit 14 reads predetermined programs stored in the ROM 16 to develop the programs in a work area in the RAM 15, and performs various processing in accordance with the programs.

More specifically, for example, the control unit 14 controls the switching elements constituting each pixel of the plane detection device 17, switches reading of the electric signal accumulated in each capacitor, and reads all the electric signals accumulated in the plane detection unit 17. The control unit 14 thus obtains image information on a radiographic image from the plane detection unit 17.

In particular, in the radiographic imaging system 1 according to the embodiment, the control unit 14 is configured to, upon radiographing, receive the patient ID number of the patient 12, radiographing ID number and radiographing conditions in connection with the radiographing, through the wireless LAN by using the console 7, and store in the RAM 15 the received patient ID number and radiographing ID number generated in each radiographing.

Moreover, to the control unit 14, a real time clock (hereinafter referred to as “RTC”) 30 as a timer unit is connected, and date and time of obtaining image information on a radiographic image is obtained as radiographing date and time. Note that the radiographing date and time may be date and time of starting reading operation by actuating the switching elements, or may be date and time of detecting irradiation through a sensor provided to detect whether irradiation has been performed or not. However, the date and time of obtaining image information is preferable, since it can be obtained most easily.

The control unit 14 is configured, as a correlating unit, to correlate the order number, patient ID number, radiographing ID number, detection device ID number and above-described radiographing conditions, which are accompanying information to accompany the image information, with the obtained image information, and to store those in the image memory 18.

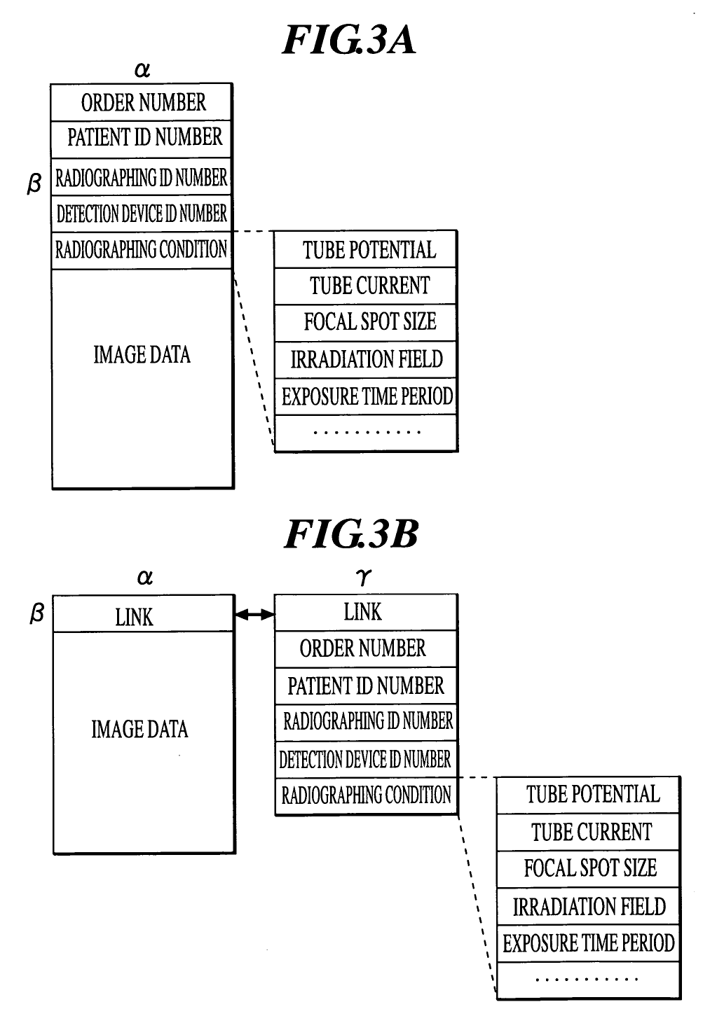

More concretely, as shown in FIG. 3A, the control unit 14 performs processing of recording the above-mentioned accompanying information to a header portion β of an image file α.

Alternatively, as shown in FIG. 3B, an accompanying information file γ containing the accompanying information and link information to link the accompanying information to the image file α is created, and link information to link the image file α to the file γ is written in the header portion β of the image file α. In this case, link information may be prepared for only one of the image file α and the accompanying information file γ. Moreover, there may be no link information in the image file α and the accompanying information file γ, and the link information may be managed in another file.

Further, the control unit 14 controls the communication unit 19, in accordance with a request from the console 7, to transmit image information on a detected radiographic image and the accompanying information correlated therewith to the console 7.

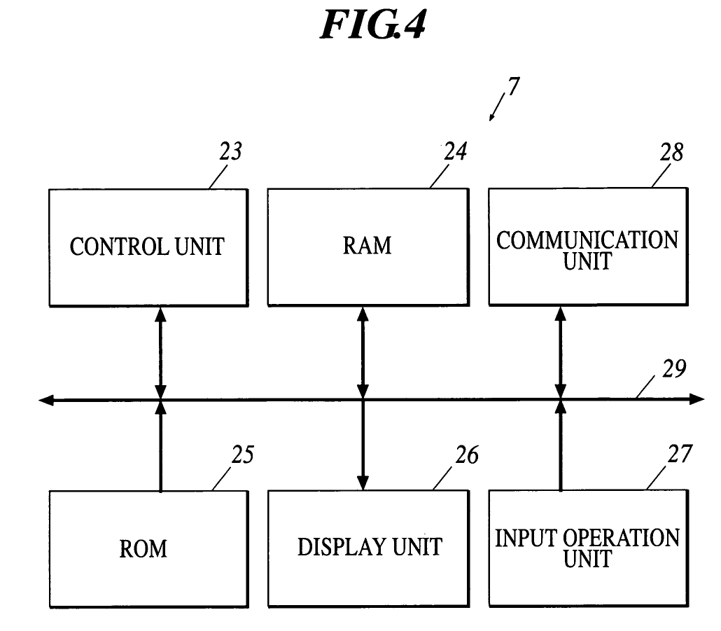

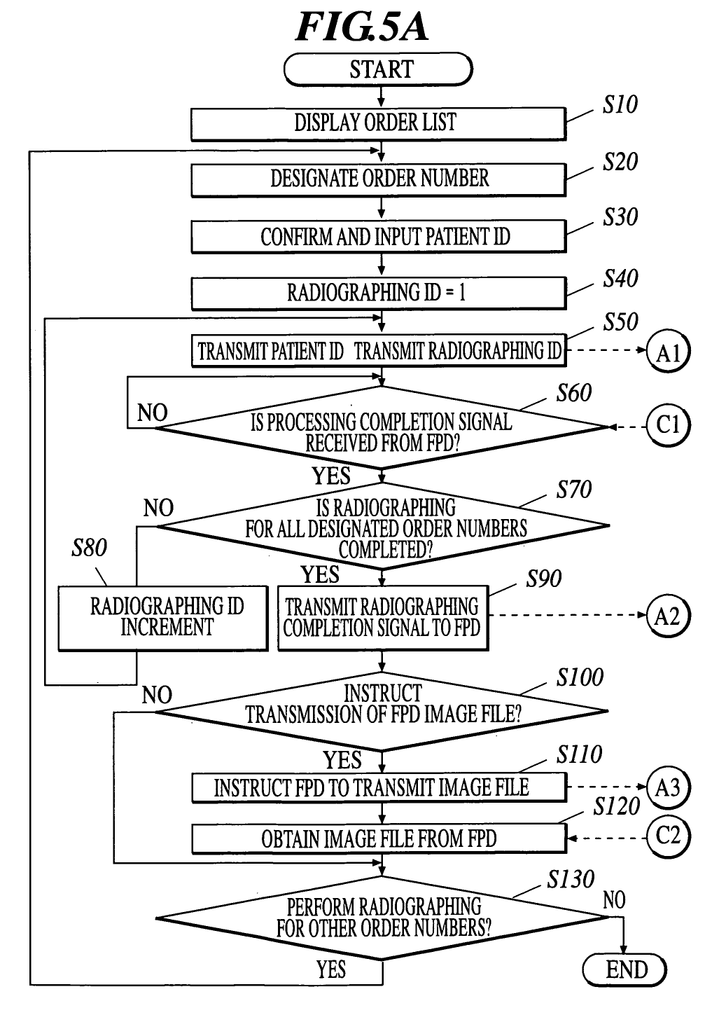

As shown in FIG. 4, the console 7 comprises a control unit 23, a RAM 24, a ROM 25, a display unit 26, an input operation unit 27, a communication unit 28, and the like, and each unit is connected to each other through a bus 29.

The display unit 26 includes, for example, such as a CRT (Cathode Ray Tube) or an LCD (Liquid Crystal Display), and displays various screens in accordance with display signals outputted from the control unit 23.

The input operation unit 27 comprises, for example, a keyboard, a mouse, and the like, and operation performed by pressing keys of the keyboard or through the mouse is detected by the control unit 23. In particular, in the radiographic imaging system 1 according to the embodiment, image information on a radiographic image detected by the FPD 6 based on predetermined operation of the input operation unit 27 is transmitted to the console 7.

The communication unit 28 communicates various information with the FPD 6 through the base station 5, by a wireless communication system such as a wireless LAN.

The control unit 23 includes a CPU (not shown) and the like. The control unit 23 reads predetermined programs stored in the ROM 25 to develop the programs in a work area in the RAM 24, and performs various processing in accordance with the programs.

In particular, the control unit 23 is configured to receive, from the server 2 through the network 8, the order information, based on which radiographing is planned to be performed by using the FPD 6 which is an operation target.

When radiographing is performed, the control unit 23 generates a radiographing ID number unique to the radiographing so that the radiographing is distinguished from other radiographing. The control unit 23 transmits the radiographing ID number and a patient ID number contained in the patient information to the FPD 6 through the base station 5 by wireless communication.

Moreover, the control unit 23 receives, from the radiographing operation device 4 via the network 8, radiographing conditions set through the radiographing operation device 4. The control unit 23 correlates the radiographing conditions received from the radiographing operation device 4 as well as radiographing conditions contained in radiographing information received from the server 2 with the radiographing ID, and stores them in a memory (not shown).

Further, the control unit 23 transmits the image information and the accompanying information correlated therewith to the server 2 via the network 8, based on operation performed through the input operation unit 27.

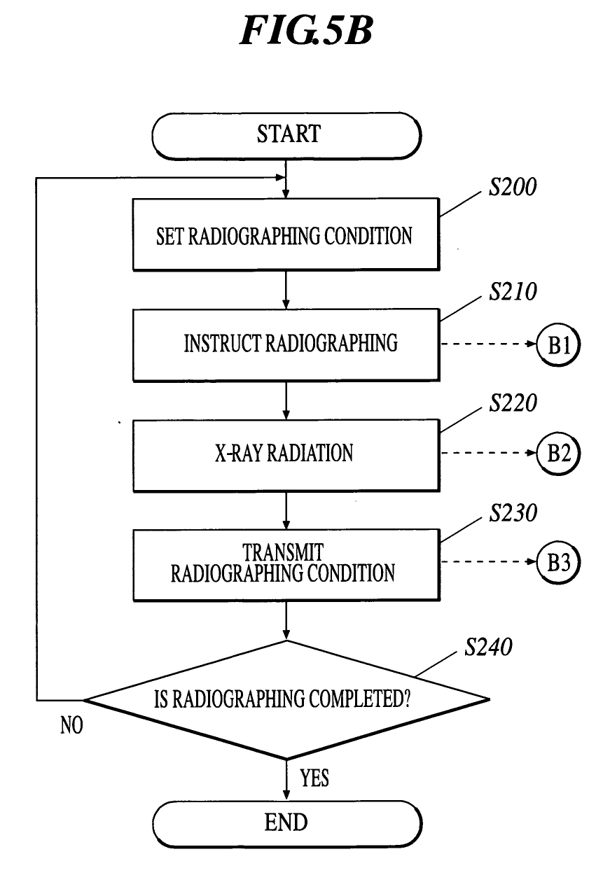

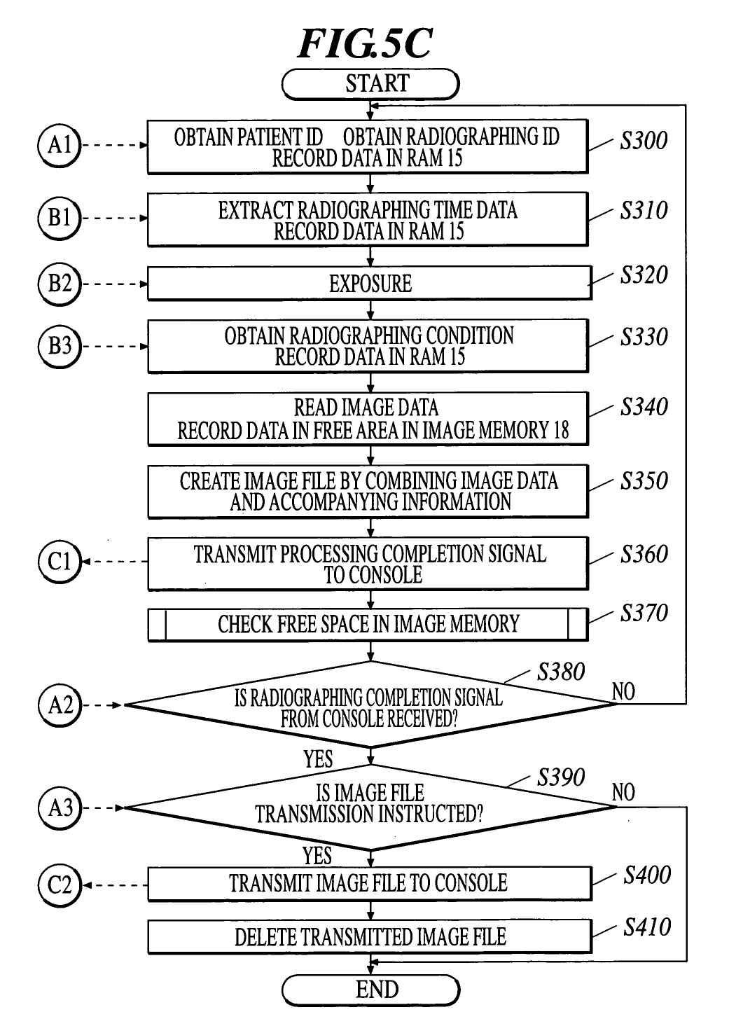

Next, referring to FIGS. 5A, 5B, and 5C, interlinking control of the console 7, radiographing operation device 4 and FPD 6 in the radiographic imaging system 1 will be explained.

Steps S10 to S130 in FIG. 5A, steps S200 to S240 in FIG. 5B, and steps S300 to S410 in FIG. 5C show flowcharts of control by each of the CPUs of the control unit 23 of the console 7, radiographing operation device 4, and control unit 14 of the FPD 6, respectively. The arrows shown by the dotted lines show communication processing between the devices.

When the radiological technologist operates the console 7 to instruct display of the list of the order information, the order information is extracted from the server 2 into the console 7, and the list of the order information is displayed on the display unit of the console 7 (Step S10).

When the radiological technologist designates through the input operation unit 27 of the console 7 an order number for radiographing (Step S20), patient information such as a patient ID is displayed (Step S30). To a radiographing ID of the patient, 1 is set as an initial value (Step S40), and the console 7 performs communication processing A1 of transmitting the patient ID and radiographing ID to the FPD 6 (Step S50). The FPD 6 obtains the transmitted patient ID and radiographing ID, and temporarily stores them in the RAM 15 (Step S300).

Meanwhile, the radiological technologist operates the radiographing operation device 4 to set radiographing conditions (Step S200). The set radiographing conditions are temporarily stored in the RAM of the radiographing operation device 4.

Subsequently, when the radiological technologist operates the radiographing operation device 4 to instruct radiographing, communication processing B1 of transmitting a radiographing instruction signal to the FPD is performed (Step S210). Upon receiving the radiographing instruction signal, the FPD 6 obtains the date and time from the RTC 30, and temporarily stores the date and time information in the RAM 15 (Step S310).

Meanwhile, the radiation generating device 3 connected to the radiographing operation device 4 radiates X-rays based on the radiographing instruction signal (Step S220). Through the X-ray radiation (processing B1), the FPD 6 is exposed (Step S320). The radiated X-rays are converted into electric signals by the plane detection unit 17 and accumulated for each pixel. When the X-ray radiation is completed, the accumulated electric signals are subjected to switching to be read, and obtained as image data.

After the X-ray radiation is completed, the radiographing operation device 4 performs communication processing B3 of transmitting the radiographing conditions stored in the RAM to the FPD 6 (Step S230). The FPD 6 obtains the radiographing conditions and temporarily stores them in the RAM 15 (Step S330).

The FPD 6 reads the image data obtained by the exposure, and temporarily stores the image data in a free area in the image memory 18 (Step S340).

The FPD 6 then combines the accompanying information such as the patient ID, radiographing ID, radiographing date and time data and radiographing conditions, which are temporarily stored in the RAM 15, the detection device ID number stored in the ROM 16, and the image data temporarily stored in the image memory 18, as described above, to create an image file α (Step S350). In this manner, processing for one radiographing ID is completed. The FPD 6 performs communication processing C1 of transmitting a processing completion signal to the console 7 (Step S360), and checks whether there is a free space in the image memory 18, that is, a free space sufficient to perform further radiographing and create an image file α continuously (Step S370).

When the console 7 receives the processing completion signal from the FPD 6 (Step S60: YES), the console 7 determines whether radiographing for the order concerned is completed (Step S70). When the console 7 has judged that the radiographing has not been completed (Step S70: NO), the radiographing ID is incremented, (Step S80), and the processing returns to Step S50 to repeat the aforementioned processing.

Although illustration is omitted, the console 7 displays the progress status of one order radiographing on the display unit 26, and the radiological technologist checks the progress status to do the work in accordance with the progress status.

In a case where further radiographing is required (Step S240: NO), the radiological technologist operates the radiographing operation device 4 to repeat the series of operation from Step S200. In a case where the radiographing is completed, (Step S240: YES), relevant operation is performed to end the processing.

Moreover, in the console 7, when it is judged that radiographing for all the designated order numbers is completed (Step S70: YES), the console 7 performs communication processing A2 of transmitting a radiographing completion signal to the FPD 6 (Step S90). When the FPD 6 receives the radiographing completion signal from the console 7 (Step S380: YES), processing from Step S390 is performed. On the other hand, when the FPD 6 does not receive the radiographing completion signal from the console 7 within a predetermined time period (Step S380: NO), further radiographing is required. The processing thus returns to Step S300 and the aforementioned processing is repeated.

Further, as processing on the console 7 side, judgment is made on whether the radiological technologist has performed operation for transmitting the image file α created by the FPD 6 to the console 7 side (Step S100). When the console 7 has judged that the operation for transmitting the image file α from the FPD 6 to the console 7 side has been performed (Step S100: YES), the console 7 performs communication processing A3 of instructing the FPD 6 to transmit the image file α (Step S110).

The FPD 6 judges whether instruction to transmit the image file α is made by the console 7 (Step S390). When the instruction is made, (Step S390: YES), communication processing C2 of transmitting the image file α stored in the image memory 18 of the FPD 6 to the console 7 (Step S400).

The console 7 receives the image file transmitted from the FPD 6 (Step S120).

In the FPD 6, the image file α which is confirmed to be received by the console 7 is deleted from the image memory 18 (Step S410). Thus, a space is created in the image memory 18 to be ready for the next radiographing.

On the other hand, when instruction to transmit the image file α is not made within a predetermined time period (Step S390: NO), the image file α is held as it is in the image memory 18 of the FPD 6, and the series of processing is completed.

Thus, when the console 7 and the FPD 6 perform communication, there may be a case where the communication between the console 7 and the FPD 6 is temporarily not possible depending on the state of the radiographing site, and the position or direction of the FPD 6. Therefore, while it is very useful that the FPD 6 itself holds the image file, when the image file held in the FPD 6 is image information only, there is a problem that the radiographing target tends to become unknown and thus it is difficult to utilize the image file for diagnosis. However, in the embodiment, since the accompanying information is correlated with the image file, the radiographing target does not become unknown even when the image file is temporarily held in the FPD 6. Accordingly, inconvenience is not caused upon utilization of the image file for diagnosis.

In particular, when the communication is wireless communication, there may be a case where it is inevitable to shut off the communication to prevent adverse effects from being caused to the other medical devices. In this case, the advantageous effect of the embodiment is remarkable.

Moreover, as processing on the console 7 side, judgment is performed on an operational input made by the radiological technologist as to whether radiographing processing for another order number is to be performed (Step S130). When radiographing processing for another order number is to be performed, the series of processing described above is repeated (Step S130: YES), and when the radiographing processing is not to be performed (Step S130: NO), the processing is ended.

The console 7 transmits the obtained image file to the server 2 as appropriate.

In the server 2, image processing such as correction processing, A/D conversion, normalization processing, and gradation processing are performed on the image data in the transmitted image file. At this time, it is possible to perform the correction in consideration of the characteristics unique to the FPD 6 on which radiographing is performed, by referring to the detection device ID number contained in the accompanying information.

Thereafter, each image data is outputted from the server 2 as appropriate, and provided as a radiographic image for diagnosis performed by a doctor. At this time, the accompanying information correlated with the image data is referred to.

As described above, according to the radiographic imaging system 1 of the embodiment, in the FPD 6, the accompanying information containing the radiographing date and time is correlated with the image information. Therefore, even when there is time difference between the time when the FPD 6 has obtained image information and the time when the image information is transmitted to the console 7, it is possible to know the accurate radiographing date and time at which the radiographic image is taken.

In the embodiment, the accompanying information includes an order number, a patient ID number, a radiographing ID number, a detection device ID number, and radiographing conditions; however, the accompanying information is not limited thereto. The accompanying information may include only one of the above, for example, only the radiographing date and time.

Moreover, for obtainment of the date and time data, although the control unit 14 of the FPD 6 is configured to include a clock function, the date and time data may be received through wireless or wired communication by using a built-in timer unit in the console 7 or the radiographing operation device 4.

Further, in the embodiment, the FPD 6 is configured to perform communication of various information with the console 7 and the radiographing operation device 4 by a wireless communication system. However, the FPD 6 is not limited thereto, and may be connected to the console 7 and the like through a predetermined cable, and configured to perform communication with each device through the cable. In particular, it is preferred that the FPD 6 is configured to be connectable to a cradle which transmits and receives various information, so that image information and the like can be transmitted to the console 7 through the cradle at an arbitrary timing.

Furthermore, in the embodiment, functions of the FPD 6 and the console 7 are realized by performing predetermined programs and the like by using the central processing unit (CPU) comprising an integrated circuit. However, the manner is not limited thereto. For example, logic circuits for realizing various functions may be used. Moreover, instruction of radiographing may be performed through the console.

The entire disclosure of Japanese Patent Application No. 2004-143574 filed on May 13, 2004 including specification, claims, drawings and abstract is incorporated herein by reference in its entirety.

1. A radiographic image detection device of a portable type to detect radiation and obtain image information on a radiographic image, comprising:
an accompanying information obtaining unit to obtain accompanying information concerning radiographing for obtaining the radiographic image; and
a correlating unit to correlate the accompanying information obtained by the accompanying information obtaining unit with the image information obtained through the radiographing.

2. The radiographic image detection device of claim 1, wherein the correlating unit comprises an image memory capable of storing a plurality of image information, and correlates each accompanying information with corresponding one of the plurality of image information stored in the image memory.

3. The radiographic image detection device of claim 2, further comprising:
a free space detecting unit to detect a free space in the image memory; and
a radiographing possibility judging unit to judge a possibility of next radiographing based on whether or not a total information amount of the image information, the accompanying information and correlation information for correlating the accompanying information with the image information exceeds the free space in the image memory detected by the free space detecting unit.

4. The radiographic image detection device of claim 3, further comprising a radiographing instruction receiving unit to receive a radiographing instruction from an external device, wherein the correlating unit correlates the accompanying information with the image information based on the radiographing instruction.

5. The radiographic image detection device of claim 4, wherein the accompanying information is radiographing date and time, and the accompanying information obtaining unit obtains the accompanying information based on the radiographing instruction, prior to obtaining the image information on the radiographic image.

6. The radiographic image detection device of claim 3, further comprising a communication unit to receive the radiographic instruction from the external device through wireless communication, wherein the communication unit receives the accompanying information prior to the radiographing.

7. A radiographic imaging system comprising the radiographic image detection device of claim 1 and a console to operate the radiographic image detection device, wherein each of the radiographic image detection device and the console comprises a communication unit to communicate with an external device, and the console receives, through the communication unit thereof, the image information and the accompanying information correlated with the image information, from the radiographic image detection device.

8. A radiographic imaging system comprising the radiographic image detection device of claim 1 and a console to operate the radiographic image detection device, wherein each of the radiographic image detection device and the console comprises a communication unit to communicate with an external device, and the console instructs, through the communication unit thereof, the radiographic image detection device to transmit the image information and the accompanying information correlated with the image information, to the console.

9. A radiographic imaging system comprising the radiographic image detection device of claim 1 and a radiographing operation device to operate the radiographic image detection device, wherein each of the radiographic image detection device and the radiographing operation device comprises a communication unit to communicate with an external device, the radiographing operation device transmits, through the communication unit thereof, a radiographing instruction signal to the radiographic image detection device, and the radiographic image detection device correlates the accompanying information with the image information based on the radiographing instruction signal.

10. The radiographic imaging system of claim 7, wherein each communication unit performs communication by using a wireless signal.

11. The radiographic imaging system of claim 8, wherein each communication unit performs communication by using a wireless signal.

12. The radiographic imaging system of claim 9, wherein each communication unit performs communication by using a wireless signal.
